(19) United States
(12) Patent Application Publication
(10) Pub. No.: US 2026/0151311 A1
(43) Pub. Date: Jun. 4, 2026

(54) COMPOSITION FOR HAIR STRENGTHENING AND DAMAGE REPAIR USING ALPHA-HYDROXY ACIDS (AHAs)

(71) Applicant: LIW PATENT COMPANY LIMITED BY GUARANTEE, Cork (IE)

(72) Inventor: Tim Meyer-Dulheuer, Cork (IE)

(73) Assignee: LIW PATENT COMPANY LIMITED BY GUARANTEE, Cork (IE)

(21) Appl. No.: 18/968,583

(22) Filed: Dec. 4, 2024

(51) Int. Cl. A61K 8/365; A61K 8/04; A61K 8/34; A61Q 5/00

(52) U.S. Cl. CPC A61K 8/365; A61K 8/046; A61K 8/342; A61Q 5/002

(57) ABSTRACT

The present invention relates to a hair repair composition and method aimed at strengthening and repairing damaged hair by targeting internal hair keratin. The invention comprises a single-step application of a composition comprising an alpha-hydroxy acid and fatty alcohol-based repair spray with a pH of ≤7, containing alpha-hydroxy acids at concentrations above 10% to promote penetration into the hair fiber. The treatment improves hair tensile strength, enhances smoothness, and provides long-lasting resilience against chemical, thermal, and environmental damage, offering an effective and cosmetically acceptable solution for hair care. This invention delivers professional-grade hair repair in a user-friendly form, maintaining hair health and strength across multiple wash cycles.

### FIELD OF THE PRESENT DISCLOSURE

The present disclosure relates to the field of hair care, specifically to a repair spray composition designed to strengthen hair and repair damage. This composition uses a unique combination of alpha-hydroxy acids at concentrations greater than 10% and fatty alcohols capable of forming covalent bonds with keratin structures in hair, thereby restoring damaged hair structure and improving its resilience.

### BACKGROUND

Hair damage from environmental factors, chemical treatments, and mechanical styling is a prevalent and ongoing concern in hair care. Everyday exposures such as UV radiation, pollution, and humidity, as well as styling practices like blow-drying, straightening, and curling, can lead to structural weakening and degradation of hair fibers. Chemical treatments such as bleaching, coloring, perming, and relaxing are particularly aggressive, as they break the disulfide bonds within the keratin structure, which can result in dry, brittle, and fragile hair with reduced elasticity and shine.

Keratin, the primary protein in hair, forms a strong fibrous structure that is essential for hair's durability and resilience. When this structure is compromised, hair loses its tensile strength, becomes rough to the touch, and shows visible signs of damage such as split ends, breakage, and a dull appearance. Repairing this damage in a way that restores the hair's internal structure rather than just the surface is a significant challenge in hair care. For instance, most hair repair products on the market are categorized as either surface conditioners or bond-building treatments.

While both aim to improve hair's appearance and strength, they have notable limitations. Briefly, surface conditioners, including leave-in treatments, masks, and oils, focus on coating the outermost layer of the hair, known as the cuticle, to improve smoothness and shine. These products use ingredients like silicones, oils, and fatty alcohols that adhere to the hair's surface and temporarily mask signs of damage.

Despite their popularity, these conditioners provide only superficial benefits that are often short-lived. They wash out easily with each shampoo, leaving hair susceptible to damage after a few washes. Moreover, repeated application can lead to buildup, which weighs down the hair and reduces its natural volume.

Similarly, bond-building products represent a more recent development in hair repair technology. These products aim to rebuild the internal disulfide bonds within hair fibers that are broken during chemical processes like bleaching and perming. By restoring these bonds, bond-builders theoretically enhance the structural integrity of hair from the inside out.

However, bond-building treatments typically involve the use of strong chemicals that can be harsh on hair if not applied under controlled conditions. Many products in this category require exacting pH levels or oxidizing agents, and they are often intended for professional salon use due to the precision required in their application. As a result, they can be inaccessible, expensive, and inconvenient for regular, at-home use.

Several patents and scientific studies have proposed methods for improving hair strength, yet they lack a comprehensive approach for durable, structural hair repair without harsh agents. For instance, U.S. Pat. No. 9,498,419 describes the use of carboxylic acids in hair coloring compositions to increase color retention and durability. Although this composition enhances the bonding of color molecules, it is not formulated for structural hair repair and does not provide the covalent cross-linking needed to restore damaged keratin fibers.

Another patent document WO 02/32383 A2 addresses hair restructuring by incorporating short-chained carboxylic acids that provide mild strengthening. However, this solution focuses primarily on surface-level restructuring and does not create robust, internal cross-links within hair keratin.

Another patent document U.S. Pat. No. 11,213,468 B2 describes a method for repairing hair using sequential application of a caustic base composition followed by an acidic crosslinking composition to enhance hair structure, optionally in combination with hair coloring or bleaching products. However, this method may not effectively create robust internal cross-links within hair keratin needed for structural repair.

In the scientific literature, “The Reactivity of Human Hair” by Wolfram et al. (Hair Research) discusses the chemical reactivity of keratin's amine and carboxylic acid groups, which are potential bonding sites for chemical treatments. However, the study does not propose a specific method or formulation that could leverage this reactivity for sustained internal repair. Ullmann's Encyclopedia of Industrial Chemistry (2012) also details various hair care ingredients and techniques but focuses on cosmetic improvements without addressing structural repair at the molecular level.

In another context, while alpha-hydroxy acids (AHAs) are widely used in skincare for their exfoliating and hydrating properties, their role in hair care has not been fully explored. AHAs, such as glycolic acid, lactic acid, and citric acid, can temporarily open the hair cuticle, allowing active ingredients to penetrate more deeply into the fiber. This makes AHAs suitable as a preparatory step in hair repair treatments, as they can enhance the uptake of subsequent agents that strengthen the internal structure.

To date, AHAs have primarily been incorporated into shampoos or conditioning treatments to increase hair's moisture retention and improve shine. However, these applications do not leverage the full potential of AHAs for structural repair when used as part of a treatment that enables deep penetration followed by bonding.

Hence, there is a requirement to address the limitations of existing hair repair products by providing a composition and method that targets internal repair rather than merely coating the hair surface, which offers a long-lasting improvement to the hair's structural integrity along with avoiding harsh chemicals and providing durable and manageable results.

### SUMMARY

In an aspect, the present disclosure generally relates to a hair repair composition and method comprising an alpha-hydroxy acid-based repair spray with a pH of ≤7, followed by adding the fatty alcohols capable of enhancing the product's conditioning, moisturizing, and protective properties while supporting the efficacy of the active repair agents.

Another aspect of the present invention is to provide a repair spray composition for hair treatment, comprising:
i. alpha-hydroxy acids at a concentration greater than 10%; and
ii. fatty alcohols; wherein pH of said composition ranges from 5-7.

In another embodiment, the present invention discloses a method for creating lasting hair repair and protection against environmental damage, involving the repair spray composition as described in the disclosure, followed by a drying step to promote bonding.

In another embodiment, the present invention provides a kit for repairing and strengthening hair comprising the repair spray composition as described in the disclosure; and an instruction manual.

Various objects, features, aspects, and advantages of the inventive subject matter will become more apparent from the following detailed description of preferred embodiments.

### DETAILED DESCRIPTION

The following is a full description of the disclosure's embodiments. The embodiments are described in such a way that the disclosure is clearly communicated. The level of detail provided, on the other hand, is not meant to limit the expected variations of embodiments; rather, it is designed to include all modifications, equivalents, and alternatives that come within the spirit and scope of the current disclosure as defined by the attached claims. Unless the context indicates otherwise, the term “comprise” and variants such as “comprises” and “comprising” throughout the specification are to be read in an open, inclusive meaning, that is, as “including, but not limited to.”

When “one embodiment” or “an embodiment” is used in this specification, it signifies that a particular feature, structure, or characteristic described in conjunction with the embodiment is present in at least one embodiment. As a result, the expressions “in one embodiment” and “in an embodiment” that appear throughout this specification do not necessarily refer to the same embodiment. Furthermore, in one or more embodiments, the specific features, structures, or qualities may be combined in any way that is appropriate.

Unless the content clearly demands otherwise, the singular terms “a,” “an,” and “the” include plural referents in this specification and the appended claims. Unless the content explicitly mandates differently, the term “or” is normally used in its broad definition, which includes “and/or.”

All processes described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

Various terms as used herein are shown below. To the extent a term used in a claim is not defined below, it should be given the broadest definition persons in the pertinent art have given that term as reflected in printed publications and issued patents at the time of filing.

### Definitions

For the purpose of the present disclosure, Alpha-Hydroxy Acids (AHAs) may be defined as Organic acids with one or more hydroxyl groups attached to the alpha carbon. Commonly used in skincare for exfoliation, AHAs, such as glycolic acid, lactic acid, and citric acid, are used in this invention for their ability to penetrate the hair cuticle and facilitate deeper absorption of subsequent treatments.

For the purpose on the present invention, Keratin may be defined as fibrous structural protein that makes up the outer layer of human skin, hair, and nails. In hair, keratin is responsible for strength, elasticity, and resilience. Damage to keratin leads to weakened hair, which this invention aims to repair by reinforcing the keratin structure.

For the purpose on the present invention, Cross-Linking may be defined as the formation of chemical bonds between polymer chains. In this context, cross-linking refers to the process of forming covalent bonds between keratin molecules in the hair, enhancing its structural integrity and resistance to damage.

For the purpose on the present invention, Tensile Strength may be defined as the resistance of hair fibers to breaking under tension. It is an indicator of hair health and structural integrity. Increased tensile strength in treated hair demonstrates the effectiveness of the repair spray in reinforcing hair structure.

For the purpose on the present invention, Cosmetically Acceptable Carrier may be defined as a formulation base that is compatible with human skin and hair, ensuring that the active ingredients can be effectively delivered without causing irritation or adverse reactions. Examples include aqueous solutions and aqueous-alcoholic solutions.

For the purpose of the present disclosure, pH Value refers to a measure of the acidity or alkalinity of a solution on a scale from 0 to 14, where 7 is neutral. In this invention, a pH of ≤7 is maintained to enhance compatibility with hair keratin and ensure stability of the active ingredients.

For the purpose of the present disclosure, Fatty Alcohols may be defined as Alcohols derived from natural fats and oils, such as cetyl alcohol, stearyl alcohol, and cetearyl alcohol. In hair care, they are used to improve the spread ability, consistency, and retention of active ingredients on hair, providing added conditioning benefits.

For the purpose on the present invention, Aqueous Solution refers to a solution in which water is the solvent. This invention uses an aqueous solution as the base for the alpha-hydroxy acid spray, which allows easy application, absorption, and effectiveness without leaving residue.

For the purpose on the present invention, Leave-In Treatment refers to a type of hair treatment that does not require rinsing after application, allowing the active ingredients to remain in contact with hair for extended periods. This invention provides a leave-in repair treatment with lasting effects on hair strength and integrity.

For the purpose on the present invention, Cosmetic Acceptability refers to the suitability of a product for use on skin and hair, encompassing factors like safety, non-irritating properties, stability, and visual appeal. Cosmetic acceptability is crucial for consumer products, ensuring that formulations do not cause adverse effects.

For the purpose on the present invention, Non-Oxidative Treatment may be defined as a hair treatment that does not rely on oxidative agents (such as hydrogen peroxide) to achieve results. The present invention is a non-oxidative treatment, reducing the risk of further damage or weakening of hair.

For the purpose on the present invention, Environmental Resilience may be defined as the ability of treated hair to withstand environmental stressors like UV radiation, humidity, and pollution without degrading. The cross-linking effect provided by this invention enhances environmental resilience, helping maintain hair quality over time.

For the purpose on the present invention, Hydration Retention may be defined as the ability of hair to retain moisture. AHAs help increase hydration retention by conditioning the cuticle, which reduces hair porosity and enhances smoothness and elasticity.

For the purpose on the present invention, Shelf Stability may be defined as the ability of a formulation to maintain its efficacy and appearance over time under normal storage conditions. Shelf stability is critical for cosmetic products, ensuring that the repair spray remains effective for a prolonged period after manufacturing.

For the purpose on the present invention, Fatty Alcohol Ester may be defined as type of compound often derived from the esterification of fatty acids and alcohols. These esters enhance the formulation's spread ability, moisturizing properties, and overall smooth feel on hair, aiding in the product's performance and user experience.

For the purpose of the present disclosure, Penetration Enhancers may be defined as Substances that increase the ability of active ingredients to penetrate the hair fiber. AHAs in this invention act as penetration enhancers, allowing subsequent agents to reach deeper within the keratin matrix.

For the purpose of the present disclosure, Nonionic Surfactants may be defined as Surface-active agents that do not carry a charge, commonly used in cosmetic formulations to stabilize emulsions and enhance the application of the product. Nonionic surfactants may be added to the repair spray to improve its performance and ensure uniform distribution across the hair.

For the purpose of the present disclosure, Conditioning Agent may be defined as an ingredient or compound that improves hair's appearance, manageability, or feel. Fatty alcohols in this invention act as conditioning agents, contributing to the product's softening effect and reducing tangling and frizz.

It should also be appreciated that the present invention can be implemented in numerous ways, including as a system, a method or a device. In this specification, these implementations, or any other form that the invention may take, may be referred to as processes.

In an embodiment, the present invention relates to a hair repair composition comprising an alpha-hydroxy acid-based repair spray with a neutral pH ranging from 5-7, along with mixing fatty-alcohols and preparing an aqueous solution capable of forming covalent bonds with hair keratin. This application enhances tensile strength, repairs damage, and improves resilience in treated hair, providing a robust solution for hair weakened by chemical or thermal treatments.

In an embodiment, the present disclosure provides a repair spray composition for hair treatment, comprising:
i. alpha-hydroxy acids at a concentration greater than 10%; and
ii. fatty alcohols, wherein pH of said composition ranges from 5-7.

In another embodiment, the alpha-hydroxy acids are selected from, but not limited to glycolic acid, lactic acid, or citric acid or a combination thereof.

In another embodiment, the fatty alcohols are selected from cetyl alcohol, stearyl alcohol, or cetearyl alcohol, enhancing the delivery and retention of active ingredients on the hair surface.

In another embodiment, said alpha-hydroxy acids are in combination with fatty alcohols as an aqueous solution.

In another embodiment, the alpha-hydroxy acids and fatty alcohols remain stable and effective over a storage period of up to 12 months under ambient conditions.

In another embodiment, the treatment with the spray improves hair tensile strength by at least 20% after a single application compared to untreated hair.

In another embodiment, said spray further comprises a cosmetically acceptable carrier selected from an aqueous medium or an aqueous-alcoholic solution, providing stability and ease of application to hair.

In another embodiment, the present invention discloses a method for creating lasting hair repair and protection against environmental damage, involving the repair spray composition as described in the disclosure, followed by a drying step to promote bonding.

In another embodiment, the present invention provides a kit for repairing and strengthening hair comprising the repair spray composition as described in the disclosure; and an instruction manual.

In an exemplary embodiment, the present disclosure may provide a method of applying an aqueous alpha-hydroxy acid-based spray with a concentration exceeding 10%, potentially in combination with fatty alcohols, at a pH of 7 or less. This step allows penetration into hair fibers, preparing the keratin for subsequent bonding.

Accordingly, this invention is designed to address the limitations of existing hair repair products by providing a composition and method that targets internal repair rather than merely coating the hair surface by strengthening hair fibers from within by creating covalent cross-links between keratin molecules. This approach offers a long-lasting improvement to the hair's structural integrity.

Nevertheless, the present invention also provides a method which utilizes a single-step application wherein AHAs is used to prepare the hair for deeper penetration. The combination of AHAs and fatty alcohols form structural bonds in hair, enhancing resilience and reducing breakage.

It is also notable that the composition disclosed herein avoids harsh chemicals and achieves hair strengthening without relying on oxidizing agents, alkalizing agents, or strong chemicals, making it suitable for regular at-home use and reducing the risk of further damage. The avoidance of harsh agents allows the composition to achieve non-damaging, long-lasting hair conditioning, offering a cosmetically acceptable solution that maintains hair health. Moreover, the composition and method disclosed herein provides durable and manageable results and said repair process leaves hair smoother, stronger, and more resistant to breakage and environmental damage. Unlike conventional treatments, the effects of this invention are retained across multiple washes.

Hence, the repair spray composition and method presented offer a significant advancement in the treatment of damaged hair by leveraging a single-step application process. This method allows for structural cross-linking within hair keratin, providing durable and resilient hair repair without the need for harsh chemicals. This invention fills a critical gap in hair care by offering a gentle yet effective solution for strengthening and repairing chemically or thermally damaged hair.

While the foregoing describes various embodiments of the disclosure, other and further embodiments of the disclosure may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

### EXAMPLES

The present invention is further explained in the form of the following examples. However, it is to be understood that the following examples are merely illustrative and are not to be taken as limitations upon the scope of the invention.

### Materials

Alpha-Hydroxy Acids (AHAs): AHAs such as glycolic acid, lactic acid, or citric acid are used at concentrations greater than 10%. These acids penetrate the hair fiber and assist in temporarily loosening hydrogen bonds, facilitating deep penetration and prepping the hair for the second application.

Fatty Alcohols: Fatty alcohols like cetyl, stearyl, or cetearyl alcohol improve the consistency and application of the spray, ensuring even coverage and enhancing retention on hair fibers.

### Example 1: Sample Preparation

Human hair samples were washed, air-dried, and then divided into groups for treatment with the one-step repair spray. The one-step repair spray may provide for a one-step application method for optimal hair repair and providing a stronger repair than multi-step products.

### Example 2: Application Process

The alpha-hydroxy acid spray is applied to the hair (wet or dry), allowing the acidic components to interact with keratin and washing the hair with water afterwards.

### Example 3: Testing Parameters

Tensile Strength: Measured using a tensiometer, comparing treated vs. untreated hair.

Durability: Hair samples underwent five wash cycles, assessing retention of strength and structural integrity.

### Example 4: Optimal pH Range

Maintaining a pH of ≤7 is critical for the stability of the formulations and ensures compatibility with the hair's natural structure, avoiding degradation and promoting repair.

### Results

Increased Strength: The sequential treatment showed a 20% improvement in tensile strength compared to untreated hair.

Durability: Treated samples-maintained strength after five wash cycles, unlike traditional conditioners.

Safety: Dermatological testing confirmed non-irritation and safety for human use.

These examples illustrate various embodiments of the invention, demonstrating its flexibility and effectiveness in treating and preventing Dermatological and joint conditions. Different formulations and administration methods cater to different user preferences and therapeutic goals.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### ADVANTAGES OF THE PRESENT INVENTION

The present invention provides a unique solution for hair strengthening and damage repair through a carefully designed, sequential application process. The following advantages make this invention stand out in the field of hair care:

### Enhanced Structural Integrity

Unlike traditional hair treatments that merely coat the surface, this invention targets the hair's internal keratin structure. The application of alpha-hydroxy acids with fatty alcohols at an optimum pH facilitates the formation of covalent cross-links within the keratin matrix. These cross-links create a durable reinforcement of hair fibers, improving resilience and significantly reducing susceptibility to breakage.

### Long-Lasting Repair and Resistance to Washing

The repair spray composition and method provide a strengthening effect that is retained after multiple washes, unlike many surface conditioners whose effects diminish after a single wash. This durability ensures that consumers experience prolonged benefits, such as stronger, smoother, and shinier hair.

Testing has shown that the treated hair retains up to 20% increased tensile strength compared to untreated hair, even after several wash cycles.

### Non-Damaging and Cosmetically Acceptable

The formulation avoids the use of harsh oxidative agents, alkalizing agents, or heavy chemicals often present in bond-building treatments. Instead, it leverages AHAs and fatty alcohols, making it a safer choice for regular use and compatible with a wide range of hair types.

The pH-balanced formula (≤7) is gentle on both hair and scalp, minimizing the risk of irritation or adverse reactions while enhancing compatibility with the hair's natural pH.

### Improved Manageability and Aesthetic Appeal

Treated hair becomes more manageable, with noticeable improvements in smoothness, shine, and softness. The cross-linking of keratin fibers reduces frizz and split ends, resulting in hair that is easier to style and maintain, even in high-humidity conditions.

The fatty alcohols included in the formulation further enhance the conditioning effect, contributing to a smooth finish and reducing fly aways without weighing down the hair.

### Environmentally Friendly Ingredients

The primary active ingredients—AHAs and fatty alcohols—are environmentally benign, offering an eco-friendly alternative to synthetic polymers and heavy chemicals. Many of these acids are derived from natural sources, aligning with consumer preferences for sustainable beauty products.

We claim:

1. A repair spray composition for hair treatment, comprising:
i. alpha-hydroxy acids at a concentration greater than 10%; and
ii. fatty alcohols, wherein pH of said composition ranges from 5-7.

2. The repair spray composition of claim 1, wherein the alpha-hydroxy acids are selected from, but not limited to glycolic acid, lactic acid, citric acid, malic acid, or a combination thereof.

3. The repair spray composition of claim 1, wherein the fatty alcohols are selected from cetyl alcohol, stearyl alcohol, or cetearyl alcohol, and wherein the fatty alcohols are configured to enhance the delivery and retention of active ingredients on a hair surface.

4. The repair spray composition of claim 1, wherein said alpha-hydroxy acids are in combination with fatty alcohols as an aqueous solution.

5. The repair spray composition of claim 1, wherein the alpha-hydroxy acids and fatty alcohols remain stable and effective over a storage period of up to 12 months under ambient conditions.

6. The repair spray composition of claim 1, wherein treatment with the repair spray improves hair tensile strength by at least 20% after a single application compared to untreated hair.

7. The repair spray composition of claim 1, further comprising a cosmetically acceptable carrier selected from an aqueous medium or an aqueous-alcoholic solution, the cosmetically acceptable carrier configured to provide stability and ease of application to hair.

8. A method for creating lasting hair repair and protection against environmental damage, involving the repair spray composition as described in claim 1, followed by a drying step to promote bonding.

9. A kit for repairing and strengthening hair comprising the repair spray composition as described in claim 1; and an instruction manual.
